(19) United States
(12) Patent Application Publication
(10) Pub. No.: US 2005/0288579 A1
(43) Pub. Date: Dec. 29, 2005

(54) Sheath

(76) Inventor: Stuart H. Miller, Clifton, NJ (US)

Correspondence Address: WELSH & FLAXMAN LLC, 2000 DUKE STREET, SUITE 100, ALEXANDRIA, VA, 22314, US

(21) Appl. No.: 11/147,390

(22) Filed: Jun. 8, 2005

Related U.S. Application Data
(60) Provisional application No. 60/577,601, filed on Jun. 8, 2004.
(60) Provisional application No. 60/578,308, filed on Jun. 10, 2004.
(60) Provisional application No. 60/579,233, filed on Jun. 15, 2004.

(51) Int. Cl. A61B 6/00

(52) U.S. Cl. 600/435; 600/585; 604/528

(57) ABSTRACT

A sheath shaped and dimensioned for reducing trauma and easing insertion thereof includes an inner sheath member, the inner sheath member including an inner sheath tip having a primary bevel and a circumferential conical surface. The sheath also includes an outer sheath member shaped and dimensioned to fit around the inner sheath member in a manner permitting relative movement. The outer sheath member includes an outer sheath tip having a primary bevel and a circumferential conical surface.

### CROSS REFERENCE TO RELATED APPLICATIONS

This application is based upon U.S. Provisional Patent Application Ser. Nos. 60/577,601, entitled “Sheath”, filed Jun. 8, 2004, 60/578,308, entitled “Sheath”, filed Jun. 10, 2004, and 60/579,233, entitled “Sheath”, filed Jun. 15, 2004.

### BACKGROUND OF THE INVENTION

1. Field of the Invention

The invention relates to a guidewire sheath. More particularly, the invention relates to a guidewire sheath having inner and outer sheath members with tips shaped and dimensioned to reduce trauma upon insertion.

2. Description of the Prior Art

In the field of interventional radiology, as well as other medical fields requiring both vascular and non-vascular access to the body, access to the interior of blood vessels must be obtained so that various devices, for example, guidewires, stents, balloons, filters and the like may be introduced into the blood vessel for medical purposes. In general, a device known as a sheath provides for desired access to the interior of the blood vessel.

A sheath generally consists of two concentric plastic tubes that are free to slide easily on each other. In accordance with current sheath designs, the inner and outer plastic tubes are in a “stepped” axial arrangement, that is, the inner plastic tube is longer than the outer plastic tube. The ends of the inner and outer plastic tubes are circular and are in a plane perpendicular to the longitudinal axis of the respective plastic tubes. This design creates a “step” at the ends of the inner and outer plastic tubes. A “step” is formed at the end of the inner plastic tube and the guidewire by the difference in the guidewire outer diameter (OD) and the OD of the end of the inner plastic tube. The length of the “step” is equal to one-half of the difference between the OD at the end of the inner plastic tube and the guidewire OD. A similar “step” is formed at the end of the outer plastic tube. The two “steps” may be of different lengths. In effect, the “steps” are merely radial discontinuities due to the diameter differences at the two “step” locations.

In use, a needle is generally used to obtain access to the interior of the blood vessel. A guidewire is inserted into the blood vessel through the needle and the needle is removed. The two plastic tubes of the sheath are then threaded over the guidewire and forced axially against the flesh of the patient until the tubes are inside the blood vessel. When the inner sheath reaches the patient, a pronounced stop of the insertion is detected by the physician. The stop is due to the radial discontinuity at the “step” between the guidewire OD and the end of the inner plastic tube. To force the inner plastic tube sheath into the hole created by the needle point, a significant axial force must be applied by the physician. The magnitude of the force depends, in part, on the geometry of the “step” and the elastic modulus of the flesh and the plastic tubes. In effect, forcing the sheath into the flesh is akin to creating an interference fit in a pair of cylinders. In an interference fit, a first cylinder is inserted within a second cylinder, wherein the second cylinder has an inner diameter that is slightly smaller than the OD of the first cylinder. Depending on the magnitude of the “step” and the elastic properties of the materials, a substantial axial force is required to produce an interference fit in this manner.

Ordinarily, with metallic materials, heating and/or cooling of the parts is generally the preferred method of assembly. When trying to insert the sheath tubes, occasionally a scalpel is used to enlarge the hole made by the needle to reduce the axial force required to insert the sheath tubes.

With the foregoing in mind, those skilled in the art will appreciate that current sheath designs have shortcomings that must be addressed. The present invention provides a sheath overcoming the shortcomings of prior sheaths.

### SUMMARY OF THE INVENTION

It is, therefore, an object of the present invention to provide a sheath shaped and dimensioned for reducing trauma and easing insertion thereof The sheath includes an inner sheath member, the inner sheath member including an inner sheath tip having a primary bevel and a circumferential conical surface. The sheath also includes an outer sheath member shaped and dimensioned to fit around the inner sheath member in a manner permitting relative movement, the outer sheath member including an outer sheath tip having a primary bevel and a circumferential conical surface.

It is also an object of the present invention to provide a sheath wherein the primary bevel of the outer sheath member extends diametrically across the outer sheath member and the primary bevel of the inner sheath member extends diametrically across the inner sheath member.

It is another object of the present invention to provide a sheath wherein the outer sheath tip is formed with an long distal end that extends further than a diametrically opposed short distal end, and the inner sheath tip is formed with an long distal end that extends further than a diametrically opposed short distal end.

It is a further object of the present invention to provide a sheath wherein the primary bevel of the outer sheath member is formed at an angle of approximately 35.81° relative to a longitudinal axis of the outer sheath member, and the primary bevel of the inner sheath member is formed at an angle of approximately 35.81° relative to a longitudinal axis of the inner sheath member.

It is still another object of the present invention to provide a sheath wherein the conical surface of the outer sheath member varies between the long distal end which has a cone angle of approximately 5.29° relative to a longitudinal axis of the outer sheath member and the short distal end which has a cone angle of approximately 35.81° relative to the longitudinal axis of the outer sheath member, and the conical surface of the inner sheath member varies between the long distal end which has a cone angle of approximately 5.29° relative to a longitudinal axis of the inner sheath member and the short distal end which has a cone angle of approximately 35.81° relative to the longitudinal axis of the inner sheath member.

It is also an object of the present invention to provide a sheath wherein it is composed of PTFE.

It is a further object of the present invention to provide a sheath wherein the inner sheath member is shaped and dimensioned to fit about a guidewire in a manner permitting relative movement.

It is another object of the present invention to provide a sheath wherein the conical surface of the inner sheath member has a minimum cone angle of between approximately 4° and approximately 60°, the conical surface of the outer sheath member has a minimum cone angle of between approximately 4° and approximately 60°, the primary bevel of the inner sheath member is formed at an angle of between approximately 30° and approximately 60°, and the primary bevel of the outer sheath member is formed at an angle of between approximately 30° and approximately 60° is between.

It is yet a further object of the present invention to provide a sheath wherein the conical surface of the inner sheath member has a minimum cone angle which differs from a minimum cone angle of the conical surface of the outer sheath member, and the primary bevel of the inner sheath member is formed at an angle which differs from an angle of the primary bevel of the outer sheath member.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of a sheath in accordance with the present invention.

FIG. 2 is a cross sectional view of the sheath shown in FIG. 1.

FIG. 3 is a cross sectional view of an outer sheath member in accordance with the present invention.

FIG. 4 is a front plan view of the outer sheath member shown in FIG. 3.

FIGS. 5 through 20 show test results comparing the present sheath to prior art sheaths.

### DESCRIPTION OF THE INVENTION

The detailed embodiment of the present invention is disclosed herein. It should be understood, however, that the disclosed embodiment is merely exemplary of the invention, which may be embodied in various forms. Therefore, the details disclosed herein are not to be interpreted as limited, but merely as the basis for the claims and as a basis for teaching one skilled in the art how to make and/or use the invention.

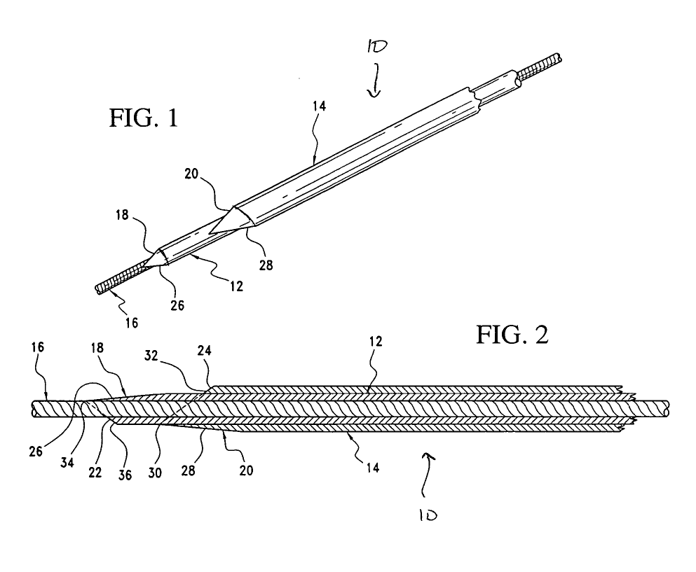

With reference to FIGS. 1 through 4, a sheath 10 in accordance with the present invention is disclosed. The sheath 10 is designed to effectively remove the undesirable “steps” encountered in conventional sheaths by using pointed, rather than blunt ends, at the ends of the inner and outer sheath members (or tubes) 12, 14. The pointed ends provide the same overall radial deformation as conventional sheath members. However, the effect of the step is greatly minimized by distributing the radial deformations along the small axial length of the point at the ends of the inner and outer sheath members 12, 14.

By using pointed ends on the inner and outer sheath members 12, 14, the magnitude of the force required to insert the inner and outer sheath members 12, 14 is greatly reduced since the total radial deformation at the “step” is spread incrementally along the length of the points on the inner and outer sheath members 12, 14. Reducing the sheath insertion force results in much better control of the sheath 10 by the physician, with subsequently less trauma encountered by the patient.

As briefly mentioned above, the sheath 10 is composed of an inner sheath member 12 shaped and dimensioned to fit around a guidewire 16 and an outer sheath member 14 shaped and dimensioned to fit about the inner sheath member 12. The inner sheath member 12 and the outer sheath member 14 are preferably formed from polytetrafluoroethylene (PTFE), for example, TEFLON, although other materials may be used without departing from the spirit of the present invention. While specific dimensions are disclosed below in describing a preferred embodiment of the present invention, those skilled in the art will certainly appreciate the guidewire, inner sheath member and outer sheath member may be made in various shapes and sizes without departing from the spirit of the present invention. The components may also be made of various materials without departing from the spirit of the present invention. What is important is that the inner sheath member 12 have an inner diameter only slightly larger than the outer diameter of the guidewire 16 such that the inner sheath member 12 fits thereabout while permitting relative movement therebetween and that the outer sheath member 14 have an inner diameter only slightly larger than the outer diameter of the inner sheath member 12 such that the outer sheath member 14 fits thereabout while permitting relative movement therebetween.

As briefly mentioned above, the inner sheath member 12 and outer sheath member 14 include pointed tips shaped to reduce trauma during insertion of the sheath 10. With this in mind, the inner sheath member 12 includes an inner sheath tip 18 and the outer sheath member 14 includes an outer sheath tip 20. As will be described below in greater detail, both the inner and outer sheath tips 18, 20 are formed with a primary bevel 22, 24 and a circumferential conical surface 26, 28.

Although specific angles, lengths and other characteristics are disclosed below in describing a preferred embodiment of the present invention, those skilled in the art will appreciate that these angles, lengths and other characteristics may be varied to suit specific applications without departing from the spirit of the present invention. As such, the equations for calculating the various characteristics are present below. In practice, these characteristics are calculated based upon the sheath members' outer and inner diameters in conjunction with the desired tip lengths and wire diameters.

With regard to the outer sheath tip 20, it is formed with a primary bevel 24 extending diametrically across the pointed tip of the outer sheath member 14. Because of the primary bevel 24, the outer sheath tip 20 is formed with a long distal end 30 that extends further than the diametrically opposed short distal end 32, with the remainder of the outer sheath tip 20 being positioned between the long distal end 30 and the short distal end 32 as the outer sheath tip 20 extends about its circular profile. In accordance with a preferred embodiment of the present invention the primary bevel 24, or bevel angle (α), is formed at an angle of approximately 35.81° relative to the longitudinal axis of the outer sheath member 14.

The outer sheath tip 20 further includes a conical surface 28, with a cone angle (β), formed about the circumference of the outer sheath tip 20. The conical surface 28 varies between the long distal end 30 that has a cone angle (that is, minimum cone angle) of approximately 5.29° relative to the longitudinal axis of the outer sheath member 14 and the short distal end 32 which has a cone angle (that is, maximum cone angle) of approximately 35.81° relative to the longitudinal axis of the outer sheath member 14. With regard to the cone angles of the outer sheath tip 20 between the long distal end 30 and the short distal end 32, it varies between 5.29° and 35.81° as the cone angle transitions from a high of 35.81° adjacent the short distal end 32 and a low of 5.29° adjacent the long distal end 30. In view of the different cone angles along the circumference of the outer sheath tip 20, the length of the outer sheath tip 20 along the conical surface 28 (that is, the cone length) changes depending upon the cone angle at that position. In particular, and considering an outer sheath member 14 having an inner radius of 0.0195 inches and an outer radius of 0.0245 inches, the outer sheath tip 20 adjacent the long distal end 30 will have a length of approximately 0.054 inches and the outer sheath tip 20 adjacent the short distal end 32 will have a length of approximately 0.00693 inches.

With regard to the inner sheath tip 18, it is also formed with a primary bevel 22 extending diametrically across the pointed tip of the inner sheath member 12. Because of the primary bevel 22, the inner sheath tip 18 is formed with a long distal end 34 that extends further than the diametrically opposed short distal end 36, with the remainder of the inner sheath tip 18 being positioned between the long distal end 34 and the short distal end 36 as the inner sheath tip 18 extends about its circular profile. Enhanced performance is achieved by positioning the long distal end 34 of the inner sheath tip 18 diametrically opposed to the long distal end 30 of the outer sheath tip 20, and similarly positioning the short distal end 36 of the inner sheath tip 18 diametrically opposed to the short distal end 32 of the outer sheath tip 20.

In accordance with a preferred embodiment of the present invention the primary bevel 22, or bevel angle (α), is formed at an angle of approximately 35.81° relative to the longitudinal axis of the inner sheath member 14. While the dimensions for the inner sheath are similar to the outer sheath for the purposes of the disclosed preferred embodiment, they may be varied to suit specific applications without departing from the spirit of the present invention. In practice, the designer will select the desired inner and outer diameters, as well as the tip lengths and wire diameters, and the remaining parameters are calculated in the manner discussed below.

The inner sheath tip 18 further includes a conical surface 26, with a cone angle (β), formed about the circumference of the inner sheath tip 18. The conical surface 26 varies between the long distal end 34 which has a cone angle (that is, minimum cone angle) of approximately 5.29° in accordance with a preferred embodiment relative to the longitudinal axis of the inner sheath member 12 and the short distal end 36 which has a cone angle (that is, maximum cone angle) of approximately 35.81° in accordance with a preferred embodiment relative to the longitudinal axis of the inner sheath member 12. With regard to the conical surface 26 of the inner sheath tip 18 between the long distal end 34 and the short distal end 36, it varies between 5.29° and 35.81° as the cone angle transitions from a high of 35.81° adjacent the short distal end 36 and a low of 5.29° adjacent the long distal end 34.

In view of the different cone angles along the circumference of the inner sheath tip 18, the length of the inner sheath tip 18 along the conical surface 26 (that is, the cone length) changes depending upon the cone angle at that position. In particular, and considering an inner sheath member 12 having an inner radius of 0.01675 inches and an outer radius of 0.0195 inches, the inner sheath tip 18 adjacent the long distal end 34 will have a length of approximately 0.054 inches and the inner sheath tip 18 adjacent the short distal end 36 will have a length of approximately 0.0038 inches.

Although a preferred embodiment of the inner sheath member 12 and outer sheath member 14 are presented above, it is contemplated the conical surface 26 of the inner sheath member 12 will have a minimum cone angle of between approximately 4° and approximately 60°, the conical surface 28 of the outer sheath member 14 will have a minimum cone angle of between approximately 4° and approximately 60°, the primary bevel 22 of the inner sheath member 12 will be formed at an angle of between approximately 30° and approximately 60°, and the primary bevel 24 of the outer sheath member 14 will be formed at an angle of between approximately 30° and approximately 60°. In addition, and as discussed above, it is also contemplated the conical surface 26 of the inner sheath member 12 may have a cone angle which differs from a cone angle of the conical surface 28 of the outer sheath member 14, and the primary bevel 22 of the inner sheath member 12 may be formed at an angle which differs from an angle of the primary bevel 24 of the outer sheath member 18.

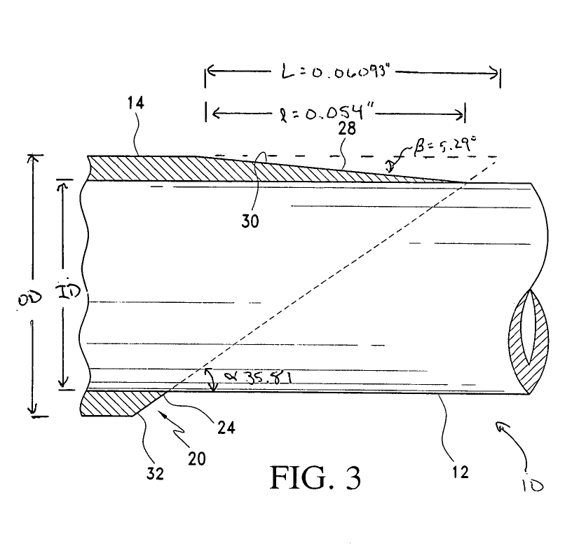

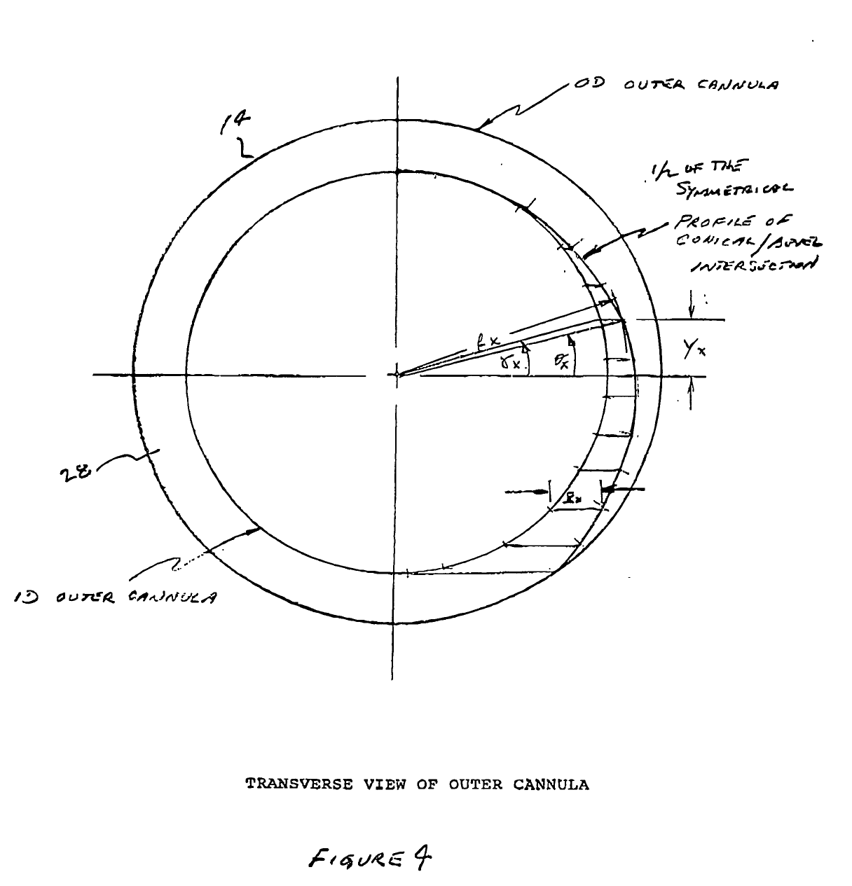

Referring to FIG. 3, the present sheath 10 employs sheath design geometry principles. The objective is to produce a sheath with tapered, sharp cutting edges which will penetrate the tissue more easily than sheaths of conventional design. As discussed above, a simple beveled point alone yields tapered surfaces along the outer sheath member which produce a force which must be overcome to insert the outer sheath member. To reduce the magnitude of this insertion force, a conical surface 26, 28, or point, is added to remove a portion of the primary bevel 22, 24. The resulting beveled/conical surface provides sharp tissue cutting edges along its entire tapered length.

In accordance with a preferred embodiment, the beveled/conical surface is generated by first grinding the primary bevel then grinding the conical surface (or reversing the grinding procedure). To prevent bending of the tip, the grinding may be controlled to provide either a feathered edge having a theoretical radial thickness equal to zero or an edge having a prescribed radial thickness and for a prescribed circumferential arc length. In production, of course, the sheath geometry would be molded.

The equations required for calculating the geometry of the tissue cutting edges are given below along with a preferred embodiment calculated in accordance with the equations. Since the calculation procedure is the same for the inner and outer sheath members, we will consider only the outer sheath design in this example. As those skilled in the art will appreciate, the parameters may be varied to suit specific applications without departing from the spirit of the present invention.

In accordance with a preferred embodiment, the outer sheath member 14 outer diameter is assumed to be 18 Ga (0.049 inches). The inner sheath member 12 outer diameter is assumed to be 0.039 inches.
R=0.0245 outer radius of the outer sheath member, inches
r=0.0195 inner radius of the outer sheath member, inches
L=0.06093 axial length of the primary bevel before conical grinding, inches
x=0.054 axial location at which the length of the cutting edge is calculated, inches

$$l := L - ( \frac{R - r}{tan ( α )} ) \qquad ( 1 )$$
l=0.054—axial length of the primary beveled after conical grinding, inches
w=0.01675—radius of the wire, inches

$$α := atan ( \frac{R - r}{L - 1} ) \qquad ( 2 )$$
α=35.810476 deg—angle of the bevel

$$β := atan ( \frac{R - r}{l} ) \qquad ( 3 )$$
β=5.290081 deg—minimum angle of the conical surface
   Rx:=r+x·tan(β) (4)
Rx=0.0245 outer radius of the conical surface at any location x, inches
   Yx:=r−x·tan(α) (5)
Yx=−0.019461 radial distance to the bevel at location x, inches

$$γ x := asin ( \frac{Yx}{r} ) \qquad ( 6 )$$
γx=−86.377504 deg angle to the inner location of the cutting edge at location, x

$$θ x := asin ( \frac{Yx}{Rx} ) \qquad ( 7 )$$
θx=−52.591841 deg angle to the outer location of the cutting edge at location, x
   Xx:=\sqrt{r²+Rx²−2·r·Rx·cos(γx−θx)} (8)
Xx=0.013651 length of the cutting edge at location x, inches
Note: the value of x varies from zero to L−(R−r)/tan(α)

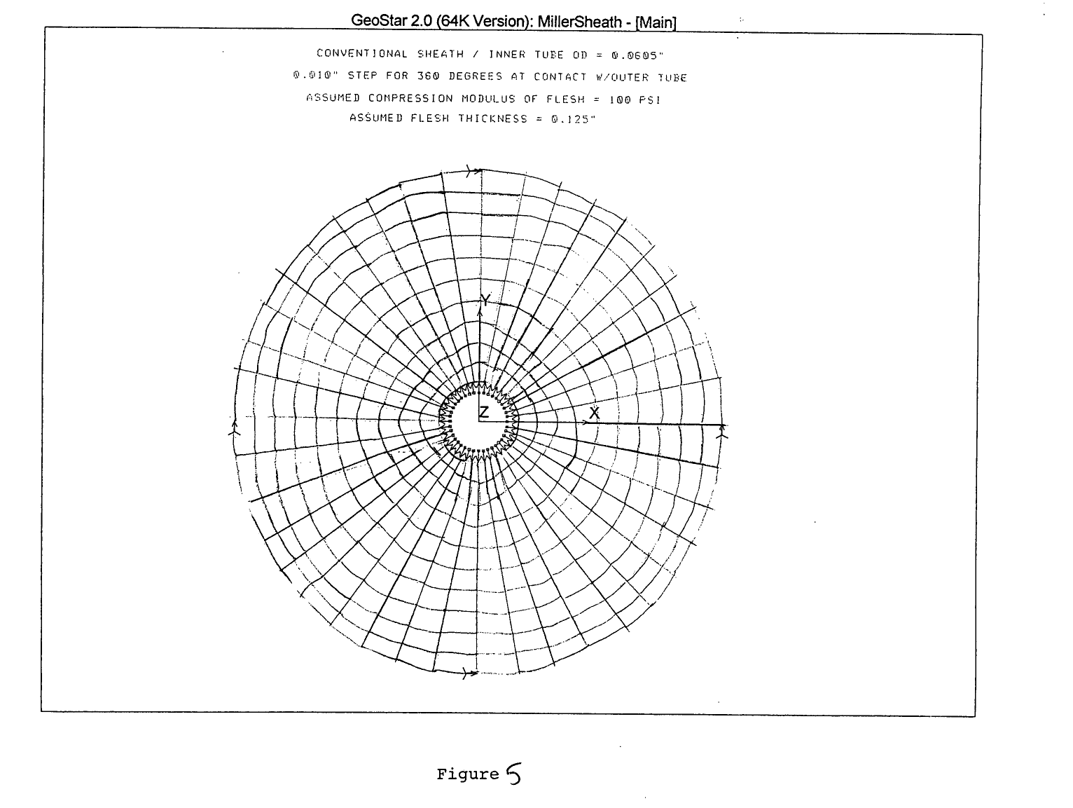

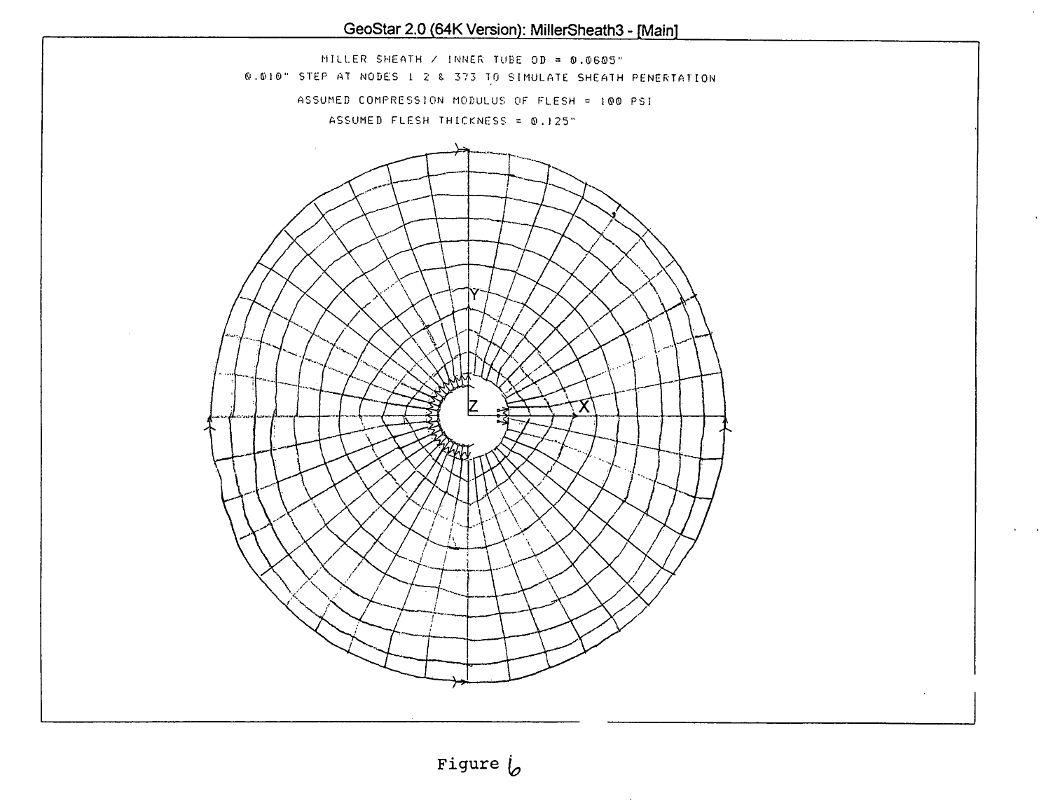

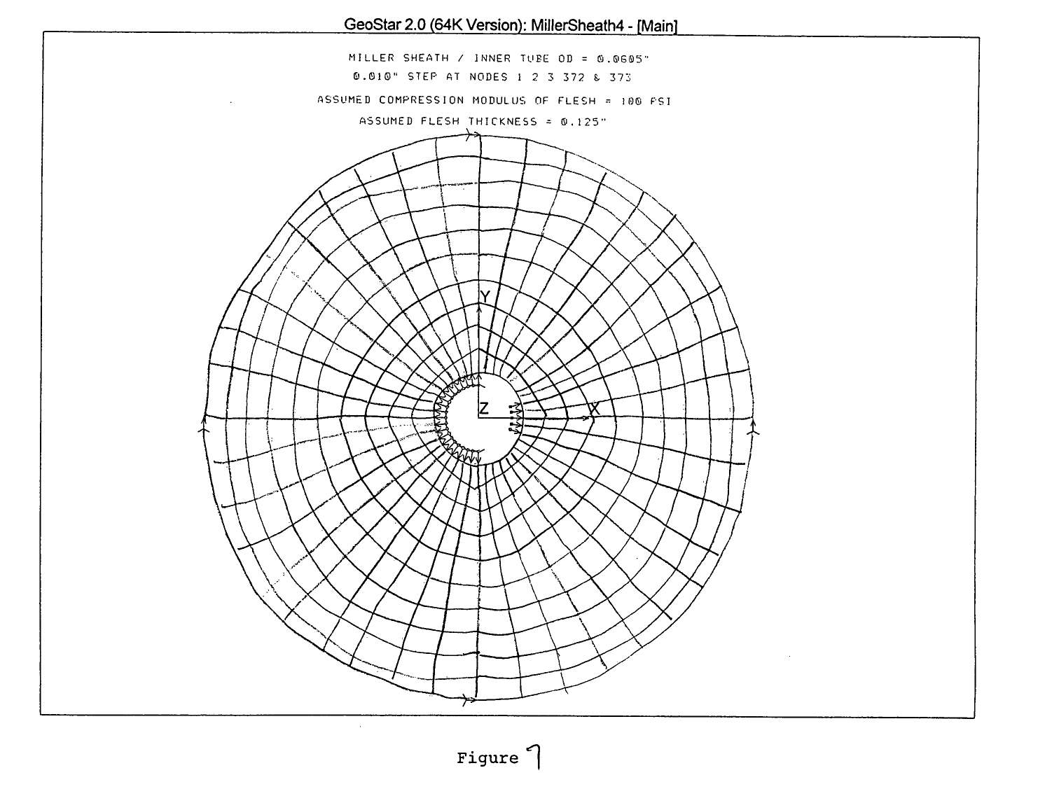

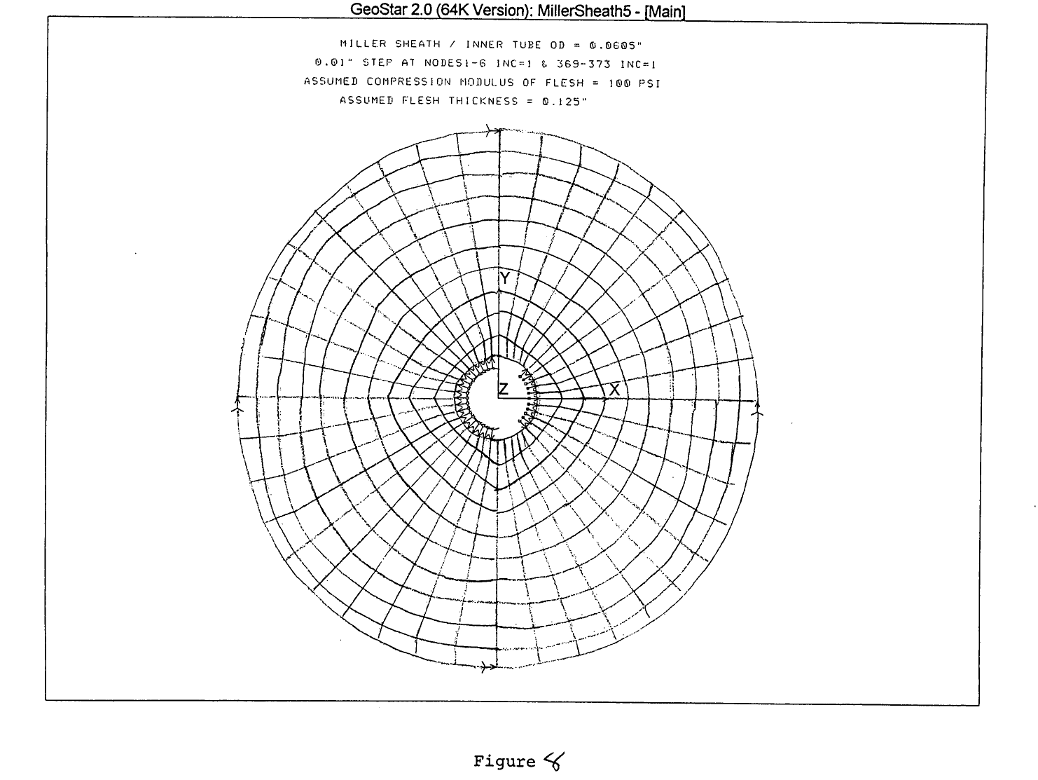

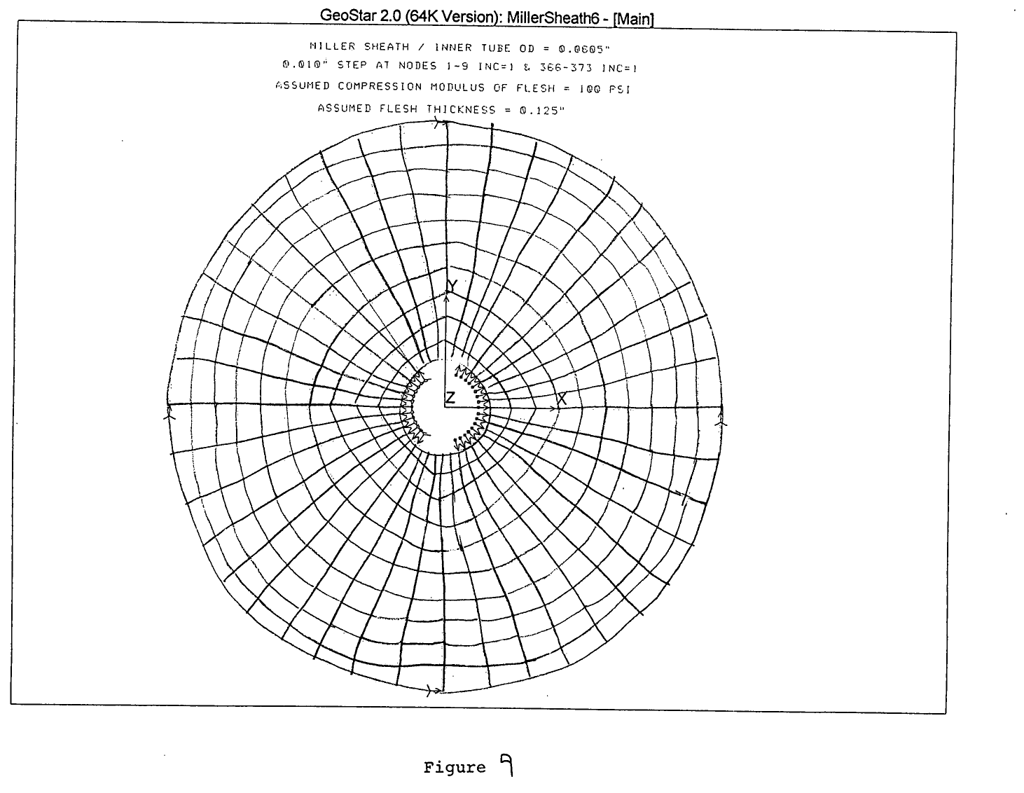

Referring to FIGS. 3 and 4, a mathematical study was conducted to determine the relationship between the design parameters shown in FIG. 3 and the cutting edge profile shown in FIG. 4. The mathematical analysis shows that the geometry of the cutting edge profile is completely determined using the given Equations. The only values to be selected by the designer are the inner and outer radii of the inner and outer sheath members 12, 14, r and R, and the lengths of the tips, L and l, and the wire outer radius, w.

In the example above, the outer sheath member 14 has an inner radius of 0.0195 inches, an outer radius of 0.0245 inches and tip lengths of 0.06093 inches and 0.054 inches. As shown in FIG. 3, this value of the cone point, l, yields a feathered edge at the point of the cone. To limit the bending of the feathered edge, the feathered edge may be blunted somewhat by removing a few thousandths of an inch off the length l. Blunting the feathered edge in this manner yields a conical surface which has an arc length sufficient to stiffen the conical tip, thereby eliminating any undesirable deformation at the conical tip. The sharpness of the conical tip is not decreased significantly.

In the example above, the value of, l, producing the feathered conical tip, is 0.054 inches. Using this value of l, the angle of the bevel, α, and the minimum angle of the conical tip, β, can be calculated using Equations (2) and (3). The outer radius to the conical point, Rx, at any location x, is given by Equation (4). The radial distance to the bevel, Yx, at location x, is given by Equation (5). The angle to the inner location of the cutting edge, γx, at location x, is given by Equation (6). The angle to the outer location of the cutting edge, θx, at any location x, is given by Equation (7). The length of the cutting edge, Xx, at any location x, is given by Equation (8).

The method for determining the profile of the intersection of the conical and beveled portions of the sheath members at any location x is as follows (see FIG. 4):
1. Draw the inner and outer diameters using a suitable scale factor.
2. Lay out the angles γx and θx.
3. Draw the portion of the radius Rx which intersects θx.
4. Draw the line Xx, between the intersections of γx and the inner radius of the cannula, r, and the intersection of θx and Rx.
5. Repeat steps (1) through (4) for all desired values of x.
6. The lines connecting the points of intersection given in steps (4) and (5) above for all selected values of x are the values of Xx, i.e., it is the cutting edge profile of the sheath.

It is contemplated either the outer sheath tip or the inner sheath tip may be stiffened by reducing the cone length at the tip of the inner and outer sheath members. This will produce a small radial step and a short arc length at the end of the conical surface defined at the inner and outer sheath tips. For example, a cone length reduction measuring thousands of an inch will increase the stiffness of the inner and/or outer sheath tips significantly.

The combined conical surface and primary bevel reduces the projected area of the beveled portion greatly when compared with merely using a beveled point on the outer sheath member. In addition, the combined conical surface and primary bevel yields a cutting edge at the outer surface of the inner and outer sheath tips, which will cut rather than tear tissue during insertion into the patient and should reduce patient trauma.

Analytical Study

An analytical study was undertaken to demonstrate how the present sheath 10 reduces the magnitude of the force required when the “steps” are encountered. This was then compared with the magnitude of the axial force required using commercially available sheaths.

The objective of the analytical study was to demonstrate the difference between the present sheath 10 and conventional, commercially available prior art sheaths. The study was conducted using Finite Element Analysis (FEA). FEA is used worldwide in the design of mechanical, fluid flow, thermal, electrical and magnetic devices. It is especially effective when making comparisons of two designs. It can be used to compare stresses, strains, deformations, reaction forces, etc. between two models having different geometries and/or mechanical properties.

FEA was used to compare the radial reaction forces developed on the inner and outer sheath members of conventional prior art designs and on the sheath members 12, 14 of the present sheath 10 since the radial reaction forces are related to the friction force required to insert the sheath.

The same FEA flesh model was used for both the conventional, commercially available prior art sheaths and the sheaths 10 produced in accordance with the present invention. The FEA model is a thin circular disk, having a central hole with a diameter equal to the outside diameter (OD) of the guidewire to simulate the first “step” in the sheath (that is, the transition between the guidewire and the inner sheath member) and a hole with a diameter equal to the OD of the inner sheath member to simulate the second “step” in the sheath (that is, the transition between the inner sheath member and the outer sheath member).

The FEA model of a conventional, commercially available prior art sheath and the present sheath 10 differed only in the manner of loading. Since the analytical procedure is the same for both the first and second “steps”, we will only describe the analysis performed on the second step.

FEA Loading

For a conventional, commercially available prior art sheath, the loading at the central hole was a prescribed radial displacement equal to one-half the difference between the OD of the inner plastic sheath member and the OD of the outer plastic sheath member. Since the ends of the sheath members are circular, and in a plane perpendicular to the longitudinal axis of the sheath member, a prescribed radial displacement was applied to all of the nodes on the central hole at the same time to simulate the abrupt loading encountered at the “step”.

As will be discussed in greater detail later, the prescribed radial displacements in the Finite Element Analysis of the present sheath 10 were applied incrementally around the central hole in the FEA model to simulate the gradual insertion of the point on the outer sheath member 14. The magnitude of the last loading step in the analysis of the present sheath 10 was the same as that in the conventional, commercially available prior art sheath, that is, 0.010″ around the entire hole to simulate complete insertion of the sheath point into the flesh.

Results of the Study

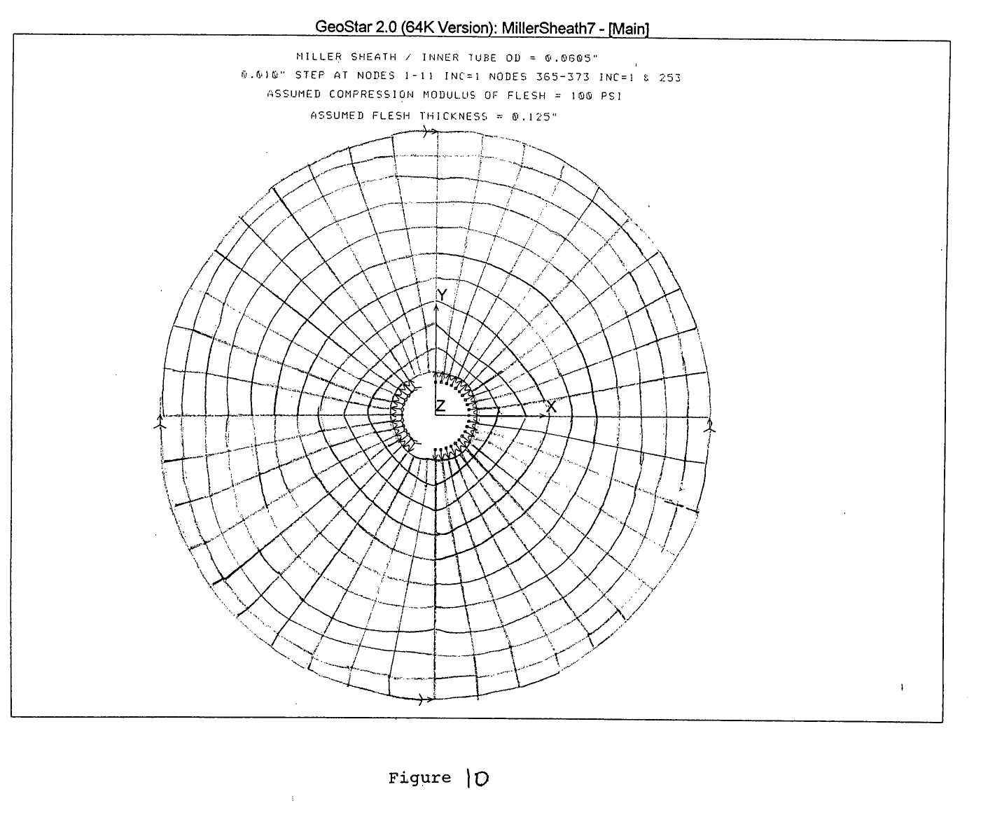

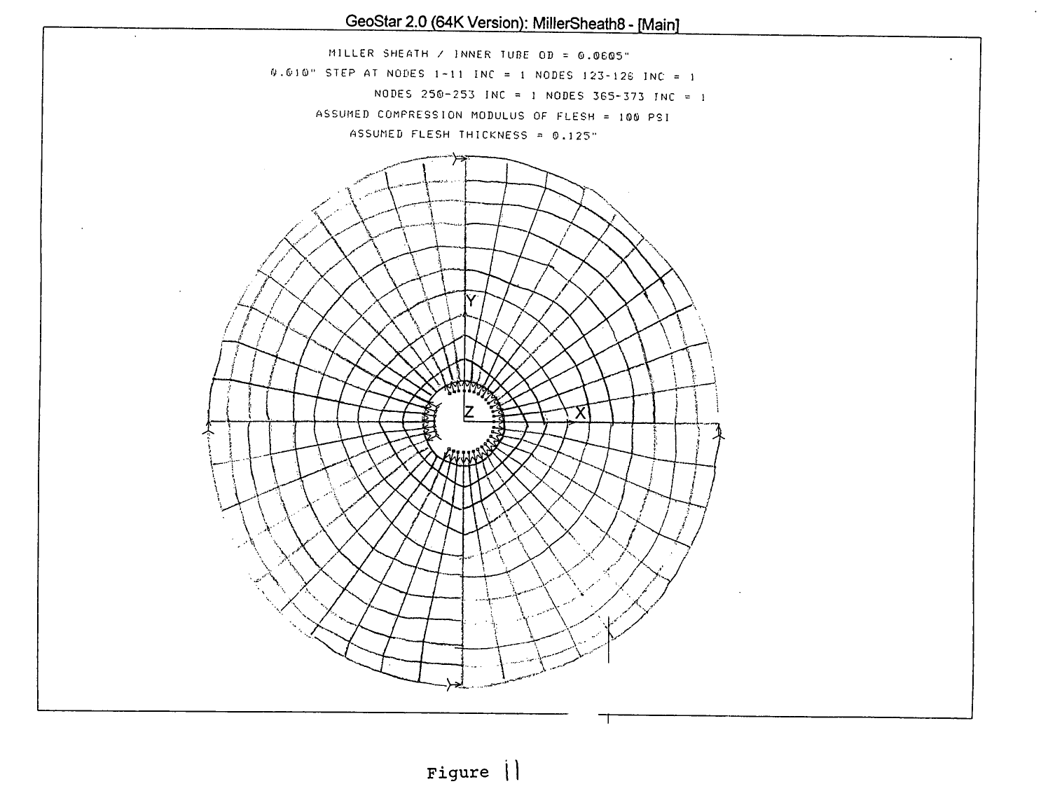

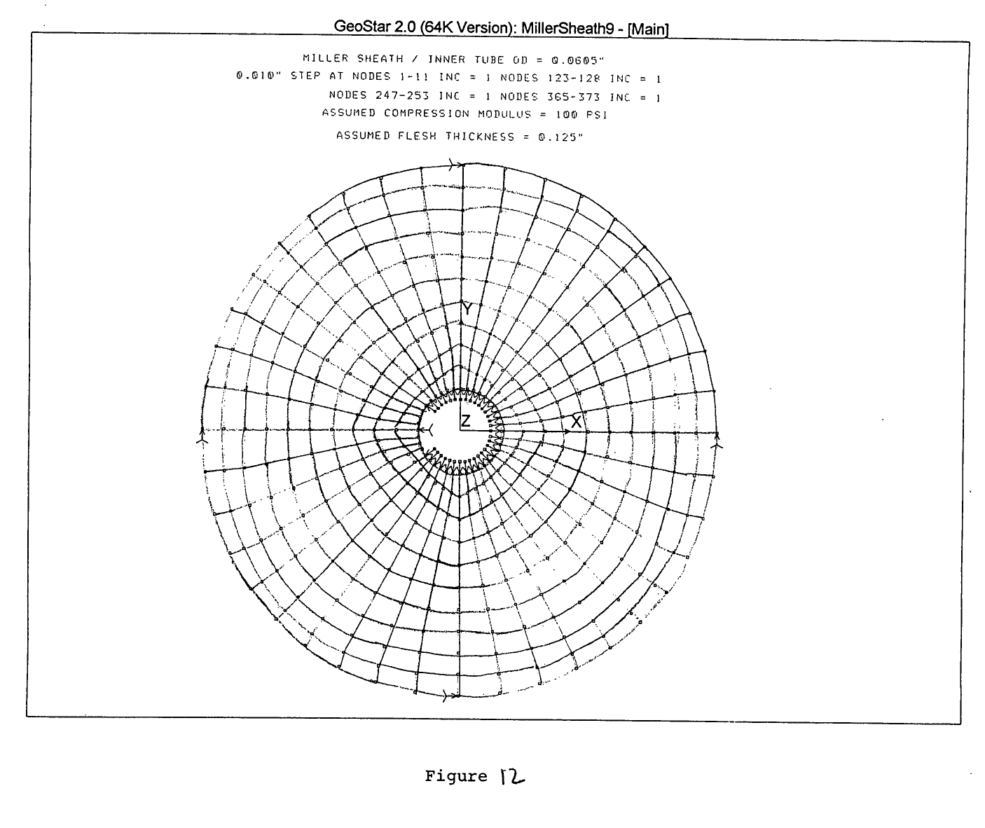

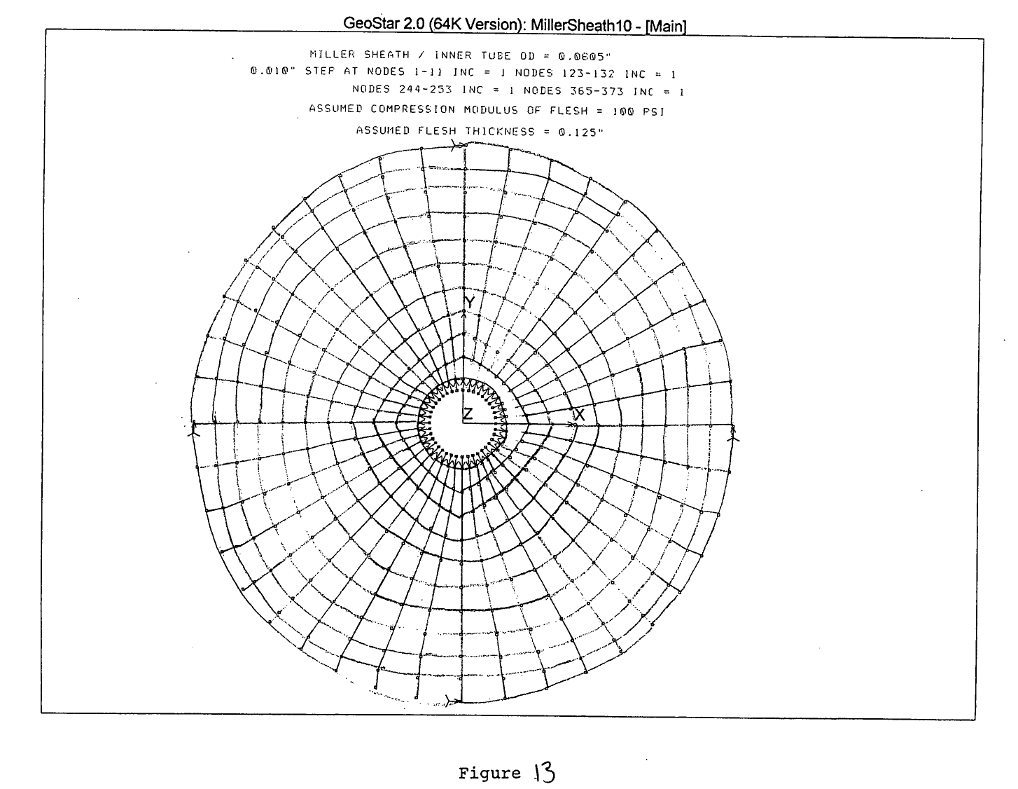

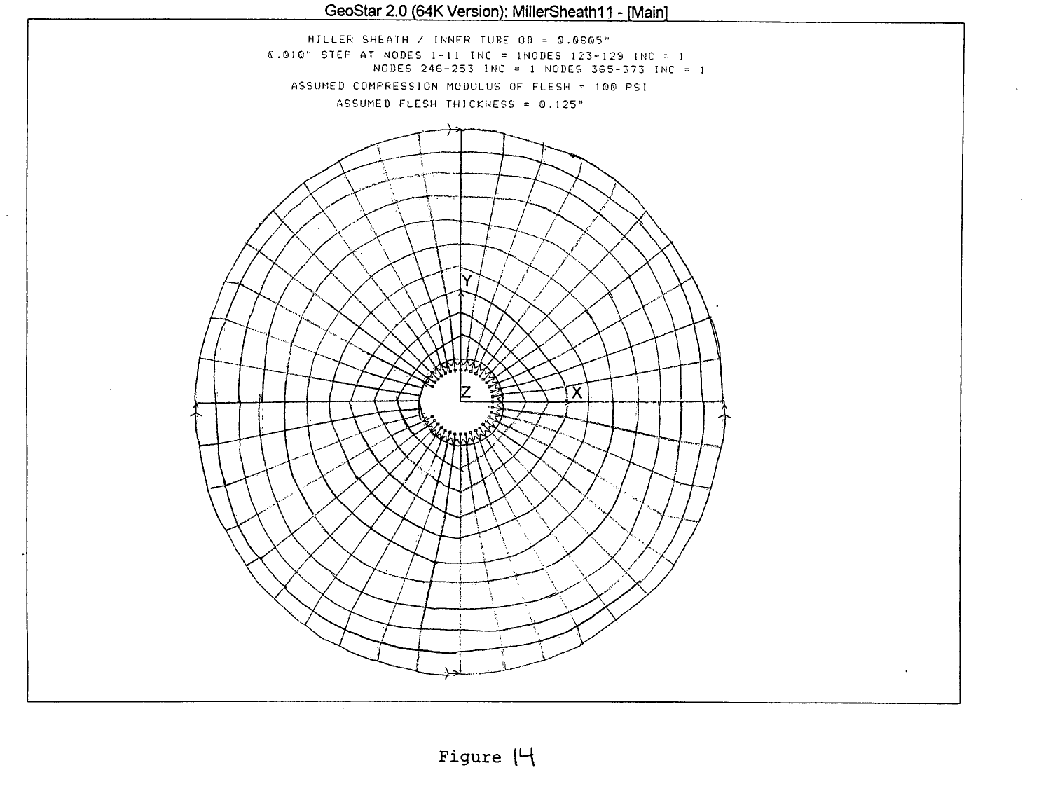

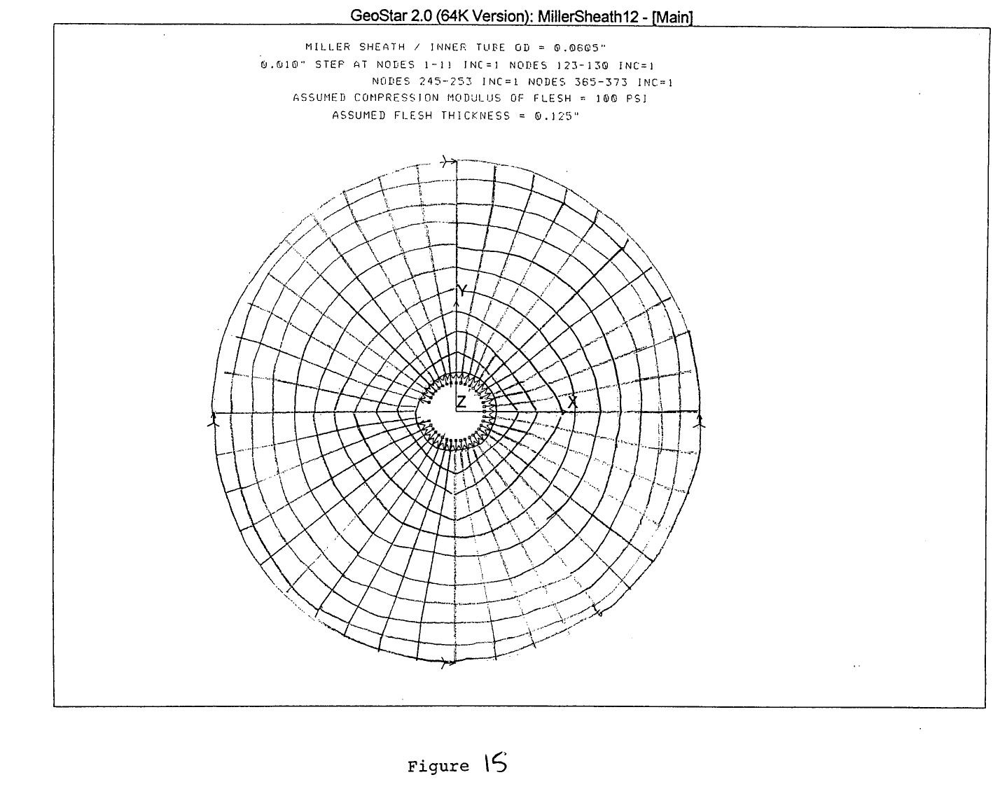

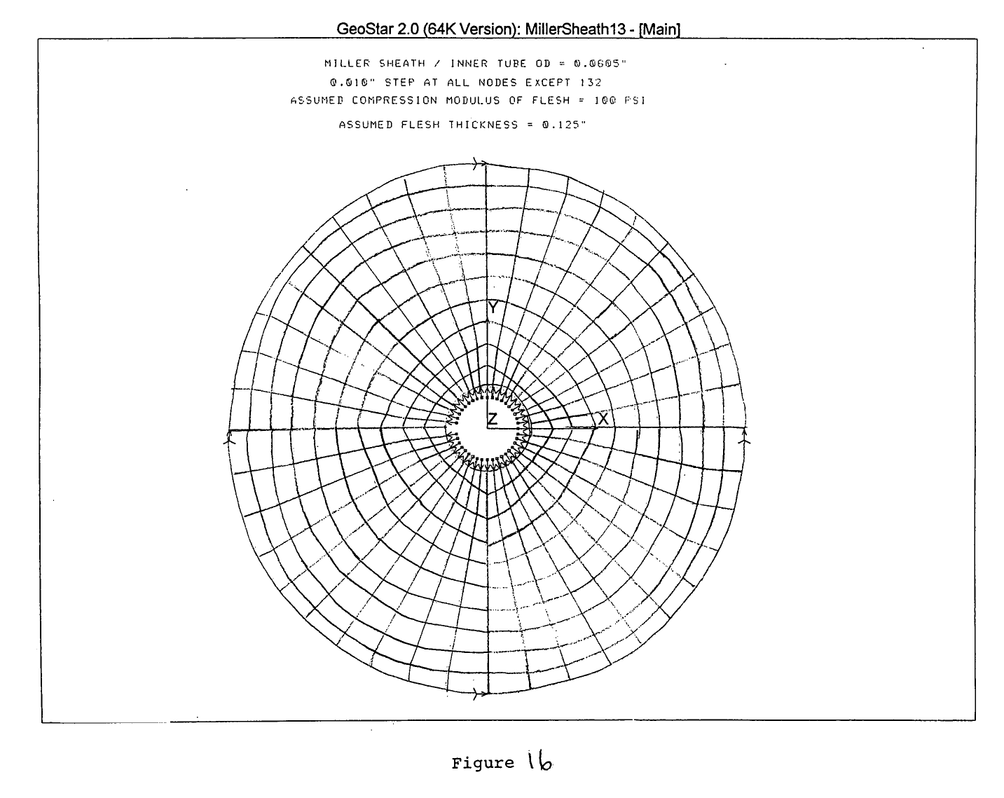

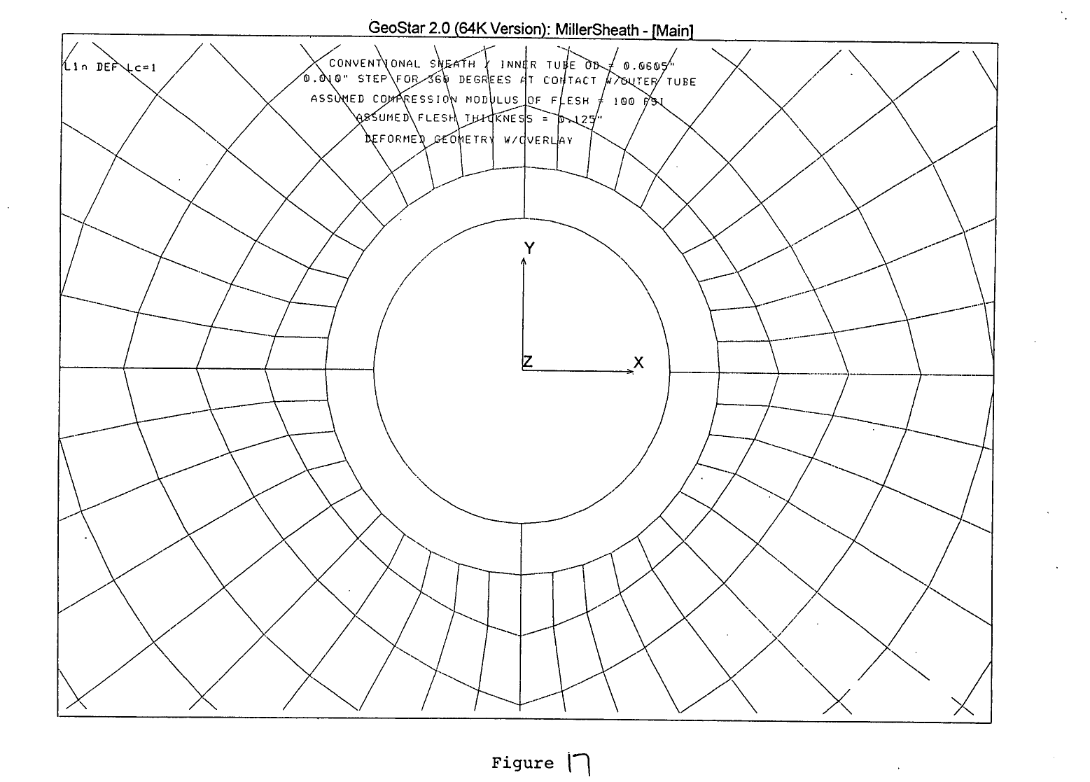

Referring to the FIGS. 5 to 20, the FEA model of a conventional, commercial prior art design is shown in FIG. 5. The loading is a prescribed, radial displacement of 0.010″ applied at all of the nodes on the hole in the model simultaneously. A compression modulus of 100 psi and a Poissons' ratio of 0.45 were used as physical properties for the flesh models.

Models designating variations of the present sheath 10 (FIGS. 6 thru 16 inclusive) show the incremental loading used in the present sheath 10 to simulate a pointed tip on the outer sheath member 14.

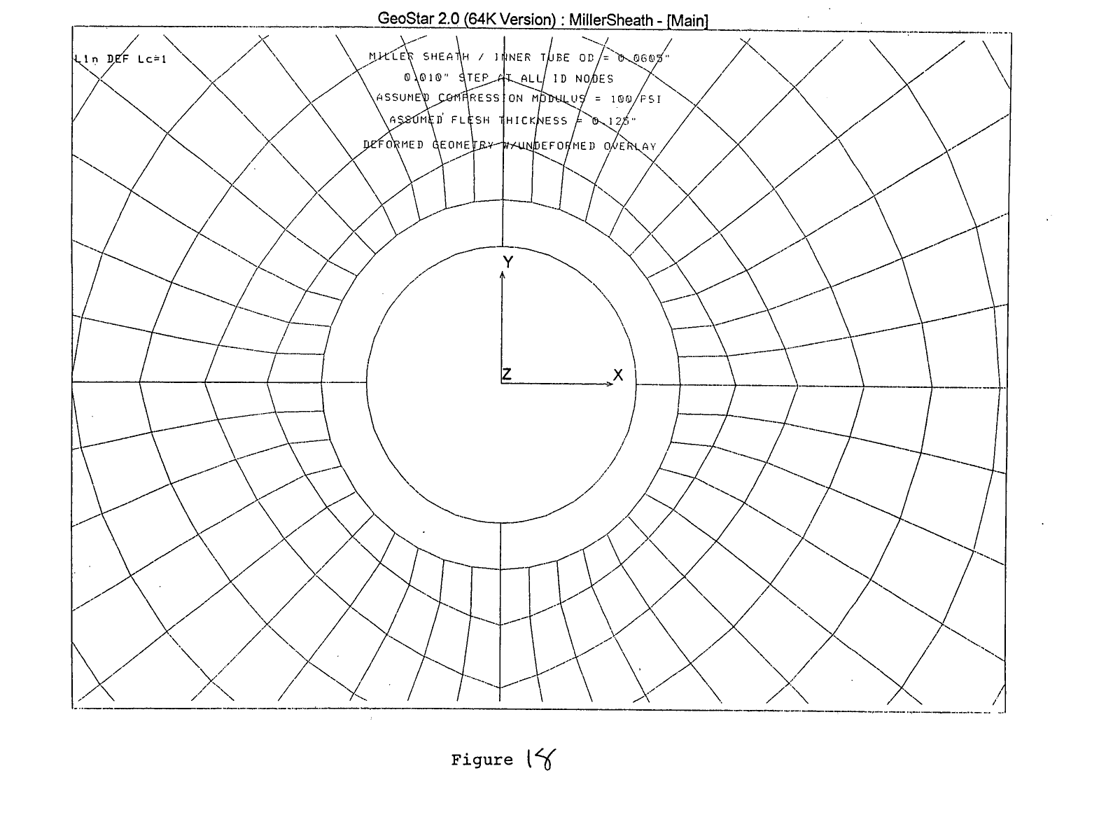

FIG. 17 shows the deformed geometry for a conventional, commercially available prior art sheath design. The circular curve at the center is the OD of the inner sheath member. The inner surrounding quadrilaterals are the deformed geometry of the flesh after a prescribed radial displacement of 0.010″.

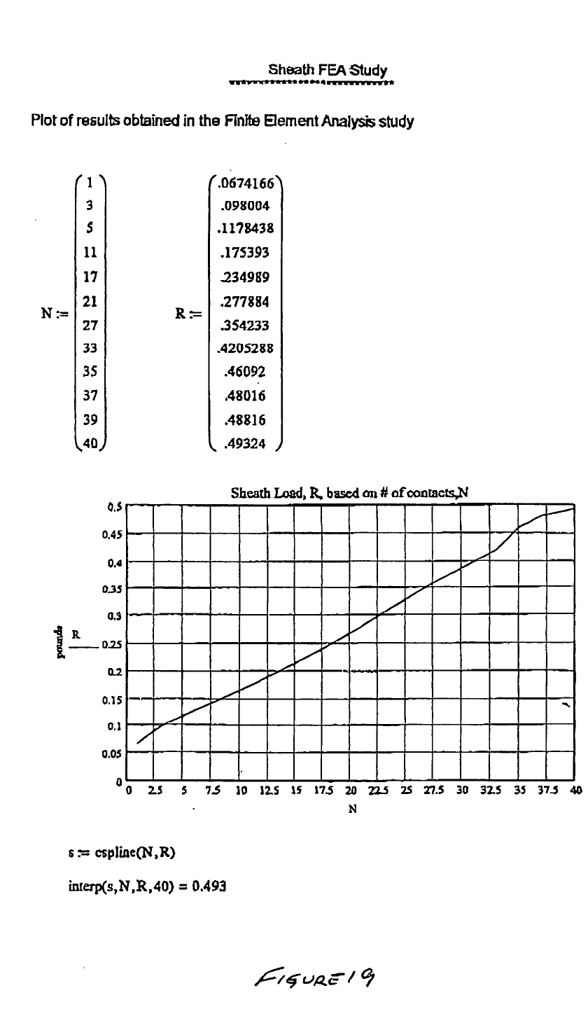

For comparison purposes, the OD of the inner sheath member 14 and the deformed geometry of the flesh for the present sheath 10 are shown in FIG. 18. Again, the inner circle is the OD of the inner sheath member 14 and the inner surrounding quadrilaterals represent the deformed geometry of the flesh when the point is inserted to a depth at which five nodes contact the OD of the outer sheath member 14.

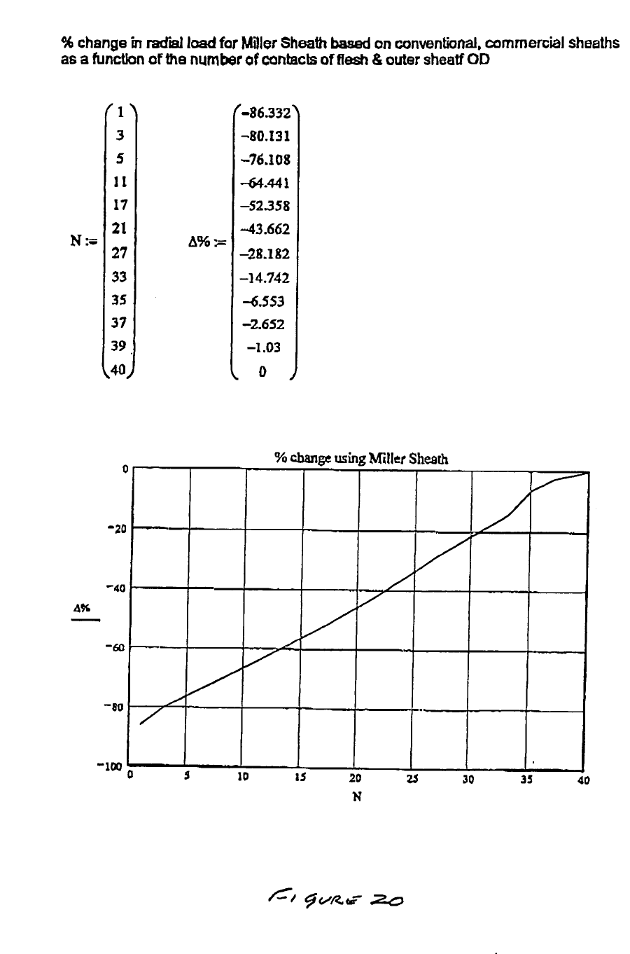

The analytical procedure was repeated for all twelve models incorporating the concepts underlying the present sheath 10 by incrementally increasing the number of nodes displaced radially by 0.010″ from 1 to 40. For each model, the deformed geometry, Von Mises stress and the radial reaction forces developed on the OD of the outer sheath member 14 and the OD of the inner sheath member 12 were calculated. The results are given in graphical form in FIGS. 19 and 20 for the present sheath 10. The radial sheath load on the OD of the inner sheath member 12 varies from 0.067# for N=1, to 0.493# for N=40 as the sheath 10 is inserted. For N=1, for example, only one node of the flesh model is displaced radially by 0.010″ by the point of the outer sheath member 14. For N=5, five points of the flesh model symmetrically located around node 1 are displaced radially by 0.010″ to simulate partial entry of the point into the flesh. For N=40, all of the points symmetrically located around Node lof the outer sheath member 14, are displaced radially by 0.010″ to simulate full insertion of the outer sheath member 14 into the flesh.

Of course, if reliable values for the coefficient of friction between the sheath members of the sheath could be determined, it would be possible to roughly calculate the axial insertion force required to overcome the friction in the system. However, the objective here is only to demonstrate that using pointed ends on the sheath members 12, 14 results in lower radial forces for the present sheath 10 than for sheaths of conventional commercial design. For comparison purposes, the total radial reaction force on the outer sheath member of the conventional commercially available sheath is 0.432#. Hence, when the sheath is inserted to the point where the outer sheath member is to be inserted into the flesh, a sudden change in the axial force is required to develop the 0.432# radial load on the outer sheath member.

In the present sheath 10, the change in radial load on the outer sheath member 14 is gradual, as shown in FIG. 19. The radial reaction force value begins at 0.067# and increases to 0.432# when the point on the outer sheath member 14 is fully inserted into the flesh. Hence, instead of encountering an initial radial reaction force of 0.432#, the present sheath 10 reduced the initial radial reaction force to only 0.067#, which is a reduction in the initial reaction force of 86.3%. The result is a sheath insertion that is much easier to control than that of conventional commercially available sheaths.

The percentage reduction in radial sheath tube loading during insertion is shown in FIG. 20, for full insertion of the present sheath 10 when compared with conventional commercially available sheath designs.

The modulus of flesh for the FEA models was assumed to be 100 psi for purposes of analysis. The comparative results between conventional, commercially available prior art sheaths and sheaths of the present sheath 10, nevertheless, should be approximated as shown herein, regardless of the flesh modulus. The pointed geometry of the present sheath 10 should always result in lower insertion forces for sheaths having the pointed, present sheath 10 regardless of the flesh modulus.

It should be noted that the point can have many different geometries, the fundamental concept being that the circular, blunt ends on the sheath members are changed to geometries which make the change from one diameter to another gradually over the length of the point.

The design of the pointed ends of the sheath members need not be “needlelike.” For example, in another embodiment of the invention, the ends of the sheath members are merely beveled at a 30 degree angle to the longitudinal axis of the sheath. The angle of the bevel at the point depends on the elastic modulus of the sheath member material. For example, relatively stiff sheath members may have pointed ends that tend to be “needlelike.” Sheath members of lower elastic modulus may merely have conical/beveled ends. The angle of the bevel depends on the elastic modulus required to prevent bending of the ends of the sheath members away from the longitudinal axis of the sheath. The design objective being to have the ends of the sheath members at an angle less than 90 degrees to the longitudinal axis of the sheath so that the pointed ends of the sheath members enter the hole in the flesh over the axial length of the bevel. Using a bevel at the ends of the sheath members forms an elliptical geometry at the ends of the sheath members. The projected length of the ellipse on the longitudinal axis determines the length of the point over which the flesh is gradually raised during insertion of the sheath.

While the preferred embodiments have been shown and described, it will be understood that there is no intent to limit the invention by such disclosure, but rather, is intended to cover all modifications and alternate constructions falling within the spirit and scope of the invention.

1. A sheath shaped and dimensioned for reducing trauma and easing insertion thereof, comprising:
an inner sheath member, the inner sheath member including an inner sheath tip having a primary bevel and a circumferential conical surface;
an outer sheath member shaped and dimensioned to fit around the inner sheath member in a manner permitting relative movement, the outer sheath member including an outer sheath tip having a primary bevel and a circumferential conical surface.

2. A sheath accord to claim 1, wherein the primary bevel of the outer sheath member extends diametrically across the outer sheath member and the primary bevel of the inner sheath member extends diametrically across the inner sheath member.

3. A sheath accord to claim 2, wherein the outer sheath tip is formed with an long distal end that extends further than a diametrically opposed short distal end, and the inner sheath tip is formed with an long distal end that extends further than a diametrically opposed short distal end.

4. A sheath accord to claim 2, wherein the primary bevel of the outer sheath member is formed at an angle of approximately 35.81° relative to a longitudinal axis of the outer sheath member, and the primary bevel of the inner sheath member is formed at an angle of approximately 35.81° relative to a longitudinal axis of the inner sheath member.

5. A sheath accord to claim 4, wherein the conical surface of the outer sheath member varies between the long distal end which has a cone angle of approximately 5.29° relative to a longitudinal axis of the outer sheath member and the short distal end which has a cone angle of approximately 35.81° relative to the longitudinal axis of the outer sheath member, and the conical surface of the inner sheath member varies between the long distal end which has a cone angle of approximately 5.29° relative to a longitudinal axis of the inner sheath member and the short distal end which has a cone angle of approximately 35.81° relative to the longitudinal axis of the inner sheath member.

6. A sheath accord to claim 1, wherein the primary bevel of the outer sheath member is formed at an angle of approximately 35.81° relative to a longitudinal axis of the outer sheath member, and the primary bevel of the inner sheath member is formed at an angle of approximately 35.81° relative to a longitudinal axis of the inner sheath member.

7. A sheath accord to claim 1, wherein the conical surface of the outer sheath member varies between a cone angle of approximately 5.29° relative to a longitudinal axis of the outer sheath member and a cone angle of approximately 35.81° relative to the longitudinal axis of the outer sheath member, and the conical surface of the inner sheath member varies between a cone angle of approximately 5.29° relative to a longitudinal axis of the inner sheath member and a cone angle of approximately 35.81° relative to the longitudinal axis of the inner sheath member.

8. A sheath accord to claim 1, wherein the sheath is composed of PTFE.

9. A sheath accord to claim 1, wherein the inner sheath member is shaped and dimensioned to fit about a guidewire in a manner permitting relative movement.

10. A sheath according to claim 1, wherein the conical surface of the inner sheath member has a minimum cone angle of between approximately 4° and approximately 60°, the conical surface of the outer sheath member has a minimum cone angle of between approximately 4° and approximately 60°, the primary bevel of the inner sheath member is formed at an angle of between approximately 30° and approximately 60°, and the primary bevel of the outer sheath member is formed at an angle of between approximately 30° and approximately 60° is between.

11. A sheath according to claim 1, wherein the conical surface of the inner sheath member has a minimum cone angle which differs from a minimum cone angle of the conical surface of the outer sheath member, and the primary bevel of the inner sheath member is formed at an angle which differs from an angle of the primary bevel of the outer sheath member.
